(19) United States
(12) Patent Application Publication
(10) Pub. No.: US 2023/0032847 A1
(43) Pub. Date: Feb. 2, 2023

(54) METHOD FOR PERFORMING MULTIPLE ANALYSES ON SAME NUCLEIC ACID SAMPLE

(71) Applicant: The Board of Trustees of the Leland Stanford Junior University, Stanford, CA (US)

(72) Inventors: Hanlee P. Ji, Stanford, CA (US); Billy Tsz Cheong Lau, Palo Alto, CA (US)

(21) Appl. No.: 17/785,882

(22) PCT Filed: Jan. 4, 2021
(86) PCT No.: PCT/US2021/012079

§ 371 Date: Jun. 15, 2022

Related U.S. Application Data
(60) Provisional application No. 62/957,594, filed on Jan. 6, 2020.

(51) Int. Cl. C12Q 1/6806; C40B 50/06; C40B 70/00

(52) U.S. Cl. CPC C12Q 1/6806; C40B 50/06; C40B 70/00; C12Q 2521/131; C12Q 2523/101; C12Q 2563/149

(57) ABSTRACT

Provided herein is a method for sample analysis. In some embodiments, the method may involve: (a) incubating a nucleic acid sample with a terminal transferase and a cyclooctene-functionalized nucleotide to produced cyclooctene-functionalized nucleic acid molecules; (b) tethering the cyclooctene-functionalized nucleic acid molecules to a tetrazine-functionalized support via an Alder cycloaddition reaction; (c) performing at least two separate primer extension reactions using the tethered nucleic acid molecules as a template to produce multiple distinct sets of primer extension products; (d) separately analyzing the sets of primer extension products using different methods to produce multiple data sets; and (e) integrating the data sets.

### CROSS-REFERENCING

This application claims the benefit of U.S. provisional application Ser. No. 62/957,594, filed on Jan. 6, 2020, which application is incorporated by reference herein.

### GOVERNMENT RIGHTS

This invention was made with Government support under grant HG000205 awarded by the National Institutes of Health. The Government has certain rights in this invention.

### BACKGROUND

Single cell sequencing has revolutionized the understanding of human biology and underpinnings of human disease. Single cell sequencing splits data contributions amongst individual cells. Combined with single molecule counting, single cell sequencing has enabled single molecule quantization of transcripts, mutations, and copy number⁵. Single cell transcriptome studies enabled the delineation of hematopoietic differentiation, discretization of transcriptional populations, and elucidated microenvironment interactions in cancer.

A diverse assortment of omic features (e.g. SNVs, gene expression, isoform usage, epigenetic modifications) play significant roles in cellular phenotypes, tissue development, and human disease. For example, genomic mutations that skip exons or frameshifts introduced at a splice site dramatically change a protein's structure. As many as 17,000 Mendelian disease-causing variants may alter mRNA structure and result in novel transcript isoforms that are pathogenic. When either investigating human disease from a primary sample or investigating a biological hypothesis, it can be valuable to characterize more than one omic feature. Each omic feature typically requires a specialized assay for interrogation. Single cell gene expression counting is typically performed using Illumina short reads, while isoform analysis is typically performed with long reads—either with Pacific Biosciences or Oxford Nanopore Technologies platforms. Significant sample splits are typically required to perform multiple experiments, which introduces dropout complexities in the measured data. Methods have been developed to integrate datasets from different omic assays across separate batches of cells, but the ideal method would be to maximize biological information by measuring omic features derived from the same cells and molecules.

The rapid development of genomic assays coupled with the scarcity of primary samples limits the amount of omic features that can be measured. Single cell transcriptome and genomic sequencing enables many applications, all of which consume sample material. Primary human samples are precious, and disease models require arduous labor to generate. Low-input assay development, sample splits, and re-amplification methods will all nevertheless deplete the source material. Conventional assays will always “destroy” samples;

the fundamental consumption of sample material remains a formidable challenge in the field of genome biology. There is a need to solve this problem using novel and creative methods.

### SUMMARY

To solve some of these challenges, a platform referred to as “attachment-based primer extension” (or “APEX”) was developed that enables perpetual re-use genetic material from a cell, e.g., a single cell. APEX involves conjugating genomic material (i.e. DNA or cDNA) to a solid phase support such as an agarose magnetic bead or the walls of a tube, and then interrogating the genetic material in multiple different ways using a non-destructive method, i.e., by copying the genetic material and analyzing the copies. FIG. 1 illustrates some of the principles of this method.

In some embodiments, the method may involve: (a) incubating a nucleic acid sample with a terminal transferase and a cyclooctene-functionalized nucleotide to produced cyclooctene-functionalized nucleic acid molecules; (b) tethering the cyclooctene-functionalized nucleic acid molecules to a tetrazine-functionalized support via an Alder cycloaddition reaction; (c) performing at least two separate primer extension reactions using the tethered nucleic acid molecules as a template to produce multiple distinct sets of primer extension products; (d) separately analyzing the sets of primer extension products using different methods to produce multiple data sets; and (e) integrating the data sets.

### BRIEF DESCRIPTION OF THE FIGURES

Certain aspects of the following detailed description are best understood when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1. Schematically illustrates some of the principles of an embodiment of the attachment-based primer extension (or “APEX”) method. 1. Rapid conjugation of cDNA analytes to a solid phase substrate enables multiple assays on diverse targets without loss. 2. Assays include whole transcriptome amplification, gene enrichment, and cellular enrichment. 3. Multiple levels of omic features can be integrated to reduce noise, and to maximize informativity of individual cDNA molecules.

FIG. 2A-C. Experimental framework. FIG. 2A: APEX conjugation. A dihydropyrazine linkage is formed between Tz-functionalized magnetic beads and TCO-tailed DNA. FIG. 2B: Experimental Procedure. APEX technology allows conjugated DNA to be subjected to many assays without loss. Magnetic beads enable the separation of reaction products from the source template. SEQ ID NO: 3. FIG. 2C: APEX applied to single cell transcriptomics. A general experimental workflow is shown for conjugating full length single cell cDNA onto magnetic beads.

FIG. 3. Single-cell transcriptome analysis utilizing APEX and nanopore sequencing. Initial feasibility experiments were performed to assess the compatibility of single-cell transcriptome analysis. (A) General experimental workflow. Single cell cDNA was generated from the 10× Genomics platform. A portion was split for standard analysis via Illumina sequencing. Another portion was conjugated to magnetic beads and cDNA amplified. The PCR products were nanopore sequenced, and the datasets were integrated. (B) Examples of spliced transcriptomic alignments with nanopore sequencing.

FIG. 4. Schematic illustration of short read and long read can be integrated bioinformatically. Full transcript structure of tagged cDNA with cellular barcode and UMI sequence specific to the 10× Genomics platform. Shown are flanking and internal adapter sequences. Short read data is processed by 10× Genomics' cellranger, yielding transcriptomic information as well as a cellular barcode ground truth. A k-mer scanning approach is used to match observed k-mers in the cellular barcodes with those observed with short reads. These are then merged with sequence alignment data.

FIG. 5. Tracking CRISPR-based edits using long reads. (A) Analysis of the MMNK1 cell line with CRISPR-based targeting of ARID1A using Illumina short reads. (B) Long read analysis detects aberrant reads at the anticipated cut site (dashed red line). (C) Cellular barcodes for the reads of interest can be mapped back to the Illumina data, yielding integrated information between datasets. (D) Target enrichment. Primers were designed for genes of interest using a custom design workflow that detects unique sequences in the genome. The boxplots show the differences in fraction of reads per cell for a selection of genes between targeted and whole transcriptome assays.

### DEFINITIONS

Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

The term “sample” as used herein relates to a material or mixture of materials, typically, although not necessarily, in liquid form, containing one or more analytes of interest. The nucleic acid samples used herein may be complex in that they contain multiple different molecules that contain sequences. Fragmented genomic DNA and cDNA made from mRNA from a mammal (e.g., mouse or human) are types of complex samples. Complex samples may have more then 10⁴, 10⁵, 10⁶ or 10⁷ different nucleic acid molecules. A DNA target may originate from any source such as genomic DNA, cDNA (from RNA) or artificial DNA constructs. Any sample containing nucleic acid, e.g., genomic DNA made from tissue culture cells, a sample of tissue, or an FFPE samples, may be employed herein.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “nucleic acid sample,” as used herein denotes a sample containing nucleic acids.

The term “target polynucleotide,” as use herein, refers to a polynucleotide of interest under study. In certain embodiments, a target polynucleotide contains one or more sequences that are of interest and under study.

The term “oligonucleotide” as used herein denotes a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers, for example. An oligonucleotide may be 10 to 20, 11 to 30, 31 to 40, 41 to 50, 51 to 60, 61 to 70, 71 to 80, 80 to 100, 100 to 150 or 150 to 200 nucleotides in length, for example.

The term “hybridization” refers to the process by which a strand of nucleic acid joins with a complementary strand through base pairing as known in the art. A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions include hybridization at about 42 C in 50% formamide, 5×SSC, 5× Denhardt's solution, 0.5% SDS and 100 ug/ml denatured carrier DNA followed by washing two times in 2×SSC and 0.5% SDS at room temperature and two additional times in 0.1×SSC and 0.5% SDS at 42° C.

The term “duplex,” or “duplexed,” as used herein, describes two complementary polynucleotides that are base-paired, i.e., hybridized together.

The term “amplifying” as used herein refers to generating one or more copies of a target nucleic acid, using the target nucleic acid as a template.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

As used herein, the term “Tₘ” refers to the melting temperature of an oligonucleotide duplex at which half of the duplexes remain hybridized and half of the duplexes dissociate into single strands. The Tₘ of an oligonucleotide duplex may be experimentally determined or predicted using the following formula Tₘ=81.5+16.6(log₁₀[Na⁺])+0.41 (fraction G+C)−(60/N), where N is the chain length and [Na⁺] is less than 1 M. See Sambrook and Russell (2001; Molecular Cloning: A Laboratory Manual, 3^{rd} ed., Cold Spring Harbor Press, Cold Spring Harbor N.Y., ch. 10). Other formulas for predicting Tₘ of oligonucleotide duplexes exist and one formula may be more or less appropriate for a given condition or set of conditions.

The term “denaturing,” as used herein, refers to the separation of a nucleic acid duplex into two single strands.

The term “genomic sequence”, as used herein, refers to a sequence that occurs in a genome. Because RNAs are transcribed from a genome, this term encompasses sequence that exist in the nuclear genome of an organism, as well as sequences that are present in a cDNA copy of an RNA (e.g., an mRNA) transcribed from such a genome.

The term “genomic fragment”, as used herein, refers to a region of a genome, e.g., an animal or plant genome such as the genome of a human, monkey, rat, fish, insect or plant. A genomic fragment may or may not be adaptor ligated. An adaptor ligated genomic fragment may have an adaptor ligated to one or both ends of the fragment, or to at least the 5′ or the 3′ end of a molecule.

In certain cases, an oligonucleotide used in the method described herein may be designed using a reference genomic region, i.e., a genomic region of known nucleotide sequence, e.g., a chromosomal region whose sequence is deposited at NCBI's Genbank database or other database, for example. Such an oligonucleotide may be employed in an assay that uses a sample containing a test genome, where the test genome contains a binding site for the oligonucleotide.

The term “adaptor” refers to double stranded as well as single stranded molecules.

A “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

If two nucleic acids are “complementary”, each base of one of the nucleic acids base pairs with corresponding nucleotides in the other nucleic acid. The term “complementary” and “perfectly complementary” are used synonymously herein.

A “primer binding site” refers to a site to which a primer hybridizes in an oligonucleotide or a complementary strand thereof.

The term “separating”, as used herein, refers to physical separation of two elements (e.g., by size or affinity, etc.) as well as degradation of one element, leaving the other intact.

The term “sequencing”, as used herein, refers to a method by which the identity of at least 10 consecutive nucleotides (e.g., the identity of at least 20, at least 50, at least 100 or at least 200 or more consecutive nucleotides) of a polynucleotide are obtained.

The term “barcode sequence”, as used herein, refers to a unique sequence of nucleotides used to identify and/or track the source of a polynucleotide in a reaction. A barcode sequence may be at the 5′-end or 3′-end of an oligonucleotide. Barcode sequences may vary widely in size and composition; the following references provide guidance for selecting sets of barcode sequences appropriate for particular embodiments: Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179; and the like. In particular embodiments, a barcode sequence may have a length in range of from 4 to 36 nucleotides, or from 6 to 30 nucleotides, or from 8 to 20 nucleotides.

The term “cell-specific barcode” refers to a sequence that is attached to a nucleic acid, where the sequence identifies the cell from which the nucleic acid or an amplification thereof is derived. Illustrated by example, the nucleic acid molecules from a first cell will be associated with a first barcode sequence, the nucleic acid molecules from a second cell will be associated with a second barcode sequence, where the barcoded nucleic acids from the different cells can be pooled. After sequencing, the sequences of the nucleic acid molecules can be tracked to a cell via the cell-specific barcode sequence appended to the nucleic acid molecules.

The term “unique molecular index” refers to a sequence that is attached to a nucleic acid molecule, where the sequence identifies the molecule from other molecules. Illustrated by example, a first molecule will be associated with a first barcode sequence, a second molecule will be associated with a second barcode sequence, where the indexed nucleic acids can be pooled, amplified and sequenced. After sequencing, the sequences of the nucleic acid molecules can be tracked to an original molecule in the sample via the index sequence appended to the nucleic acid molecule.

The term “enzymatically attaching”, as used herein, refers to the covalent addition of a reactive group onto a nucleic acid using an enzyme. Such an addition may be done using a ligase, terminal transferase, polymerase, or another enzyme that is capable of attaching an oligonucleotide or a nucleotide that contains the reactive group onto the nucleic acid. In attaching the reactive group, the reactive group is unmodified during the addition.

The term “covalently reacting”, as used herein, refers to a chemical reaction in which new covalent bonds are formed between two different moieties.

The term “click chemistry” and “Alder cycloaddition” as used herein, refer to a specific chemistry for joining compounds, particularly biopolymers, together. Click chemistry includes [3+2] cycloadditions, such as the Huisgen 1,3-dipolar cycloaddition, e.g., the Cu(I)-catalyzed stepwise variant (see Spiteri et al. Angewandte Chemie International Edition 2010 49: 31-33, thiol-ene click reactions, the Diels-Alder reaction and inverse electron demand Diels-Alder reaction, [4+1] cycloadditions between isonitriles (isocyanides) and tetrazines, nucleophilic substitution especially to small strained rings like epoxy] and aziridine compounds, carbonyl-chemistry-like formation of ureas, and addition reactions to carbon-carbon double bonds like dihydroxylation or the alkynes in the thiol-yne reaction. One click chemistry of particular interest includes the azide alkyne Huisgen cycloaddition using a copper (or another metal such as ruthenium or silver) catalyst at room temperature. Click chemistry, including azide-alkyne cycloaddition, is reviewed in a variety of publications including Kolb et al (Angewandte Chemie International Edition 2001 40: 2004-2021), Evans (Australian Journal of Chemistry 2007 60: 384-395) and Tornoe (Journal of Organic Chemistry 2002 67: 3057-3064).

The terms “reactive group” and “reactive site” are used herein to distinguish between the two moieties that can react with one another to produce a covalent bond (e.g., in the click chemistry described above) between to elements. For the purposes of this disclosure, the “reactive group” is the group that is present on the nucleic acid whereas the “reactive site” is present on the porous support. However, it is understood that in some cases, the reactive group can be a first moiety (e.g., an alkyne) and the reactive site can be an a second moiety that specifically reacts with the first moiety (e.g., an azide) whereas in other cases the reactive group can be the second moiety (e.g., an azide) and the reactive site can be the first moiety (an alkyne). Notably, the reaction that occurs between a reactive group and a reactive site does not affect ability of the nucleic acid to base pair with other complementary sequences.

The term “support”, as used herein, refers to membranes, beads, as well matrices that include cross-linked polymers. The wall or bottom of a tube is a type of support. In some cases, a support may be made from a sugar- or acrylamide-based beads having a diameter of 10 μm to 500 μm (e.g., 25 μm to 250 μm) that are produced in solution (i.e., in hydrated form). Beads may be supplied as wet slurries that can be easily dispensed to fill and pack a column of any size. Such beads are extremely porous and sufficiently large to allow nucleic acid to flow as freely into and through the beads as they can between and around the surface of the beads. Other porous supports include membranes, e.g., silica membranes, and are commonly used to purify nucleic acids.

The term “covalently tethering”, as used herein, refers to an action that results in a first element, e.g., a nucleic acid, being joined to a second element, e.g., a support, by a covalent bond. Covalently tethering may be direct or indirect (e.g., via another molecule, e.g., an oligonucleotide of another type of linker) that is covalently added either of the first element or the second element.

The term “free in solution,” as used here, describes a molecule, such as a primer extension product, that is not bound or tethered to another molecule.

The term “primer extension reaction”, as used herein, refers to the extension of a primer by the addition of nucleotides using a polymerase. If a primer that is annealed to a nucleic acid is extended, the nucleic acid acts as a template for extension reaction.

The term “primer extension products”, as used herein, refers to the products of a primer extension reaction.

The term “eluting”, as used herein, refers to the liquid phase separation of a product (e.g., a primer extension product) from a support. In most cases, the product that is eluted is collected, e.g., in a vessel.

The term “gene specific primer”, as used herein, refers a primer that is designed to hybridize to a single target sequence in the genome of an organism under study. In certain cases, the target sequence may have been duplicated, in which case a gene specific primer may hybridize to multiple sequences, where each of the multiple sequences is a duplicate of another. In many cases, a gene specific primer may bind upstream of a sequence of interest, where a sequence of interest may have a role in a disease or condition, and may be polymorphic (e.g., may contain a potential mutation or SNP), and extension of the gene specific primer may produce a copy of the sequence of interest.

The term “universal primer”, as used herein, refers to a primer that is designed to bind to all of the nucleic acid molecules in a sample.

The term “spin column”, as used herein, refers to a chromatography column that is designed to a sample, wash and elution solvents (and optionally other liquids such as a reaction mix) can be added to the column and retained in the column until a centrifugal force (e.g., an RCF in the range of e.g., 50 to 15000, e.g., 50 to 500, depending on the type of support) is applied to the column. RCF may be estimated by the following formula RCF=1.12r(rpm/1000)², where r is the radius of the rotor used.

The term “integrating”, as used herein, refers to the linking of two or more data sets such that multiple data points for a single cell or single molecule, as derived from different data sets, are linked, can be combined as one, and can be viewed together. Other definitions of terms may appear throughout the specification.

### DETAILED DESCRIPTION OF EXEMPLARY EMBODIMENTS

As noted above, in some embodiments the method may involve: (a) incubating a nucleic acid sample with a terminal transferase and a cyclooctene-functionalized nucleotide (e.g., 5-trans-Cyclooctene- PEG₄-dUTP) to produce cyclooctene-functionalized nucleic acid molecules; (b) tethering the cyclooctene-functionalized nucleic acid molecules to a tetrazine-functionalized support (e.g., tetrazine-functionalized magnetic agarose beads or tube) via an Alder cycloaddition reaction; (c) performing at least two separate primer extension reactions using the tethered nucleic acid molecules as a template to produce multiple distinct sets of primer extension products; (d) separately analyzing the sets of primer extension products using different methods to produce multiple data sets; and (e) integrating the data sets.

In some embodiments, the nucleic acid or a copy thereof can be added using any click reaction (not necessarily the reaction described in the examples) e.g., via a DBCO/azide reaction. Further, there are other ways to produced click-reactive nucleic acids, other than adding modified nucleotide using terminal transferase. For example, the click reactive nucleotide could be added to the 5′ end of the nucleic acid. In some embodiments the nucleic acid may be copied or amplified using a primer that has a click-reactive nucleotide.

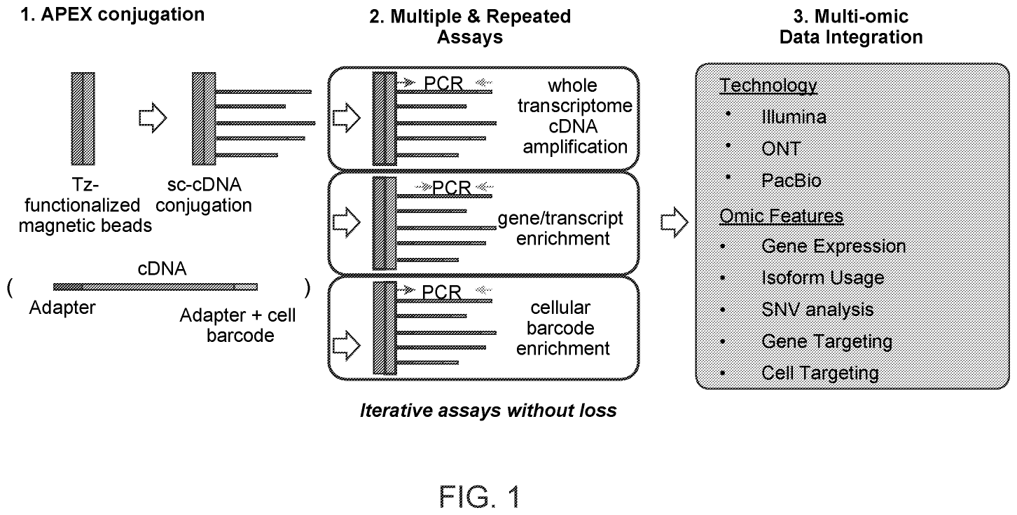

In this method, the original molecules (DNA or cDNA) are retained, and only copies of those molecules are analyzed, e.g., by sequencing. For example, as illustrated in FIG. 1, the at least two separate primer extension reactions may include whole transcriptome cDNA amplification (by PCR), amplification of one or more genes or transcripts, or amplification of the nucleic acid from one or more cells using primers that hybridize to a barcode, for example. This feature enables multilevel, iterative analysis of omic features from the same sample, without splitting the sample and without sample loss. The method is particularly valuable for limited samples, i.e., samples that have low amounts of nucleic acid. Such analysis is largely impossible using conventional analysis methods because conventional analysis methods consume the sample. In the present method, multiple rounds of single cell analysis (e.g. whole transcriptome, targeted gene sets, and targeted cell populations) can be applied to the same originating analytes and measured on sequencing platforms best suited to them (e.g. read counting on short-read platforms (e.g., on the Illumina platform), and isoform usage on long-read platforms, e.g., on the ONT platform). For data integration and denoising, the data obtained from multiple assays can be linked by discrete features such as cellular barcodes as found in single cell sequencing, or by genotypes such as SNVs or CRISPR edits, where data integration allow one to view multiple data sets for the same cell and/or molecule. Uniquely, this method allows one to integrate many different types of genomic readouts, thereby providing a way to analyze the same source molecule in multiple different ways. For example, the method can be used create and analyze individual molecules and individual cells that have been analyzed in variety of different ways, where the different data sets are integrated (e.g., on a cell-by-cell and/or molecule-by-molecule basis). Multiple interrogation of omic features with this method creates a unique platform for information maximization (through data integration) and noise minimization (through replicate experiments and imputation) by repeatedly measuring from the same cells and molecules.

In some embodiments, the method may be done on a cell-by-cell basis by, e.g., isolating single cells, adding different cell-specific barcodes to the nucleic acid of the different cells, and analyzing the pooled, barcoded nucleic acid from those cells. In these embodiments, the nucleic acid sample of step (a) may be made by: (i) compartmentalizing a population of cells into single cell compartments; (ii) making cDNA and/or genomic DNA libraries from the cells in the compartments, wherein the cDNA and/or genomic libraries are tagged with different cell-specific barcodes; and (iii) pooling the cDNA and/or genomic libraries made from the cells. In these embodiments, the integrating of step (e) may be done on a cell-by-cell basis, i.e., such that two or more data sets relating to a single cell can be matched and analyzed together. This may be done by partitioning single cells with single particles (i.e., beads) that are linked to oligonucleotide primers. Such partitioning may be done using droplets (using, e.g., 10× Genomics Chromium system) microwells (using, e.g., Becton, Dickinson RHAPSODY™ method), microfluidic chambers, or patterned substrates for example. Methods for performing analysis of DNA and cDNA on a cell-by-cell are known (see, e.g., Porubsky et al, Nat Commun. 2017 8: 1293; Fan et al, Science 2015 347: 1258367; Fu et al, Anal Chem. 2014 86: 2867-70; Fu et al Proc Natl Acad Sci U S A. 2014 111: 1891-6; Petti et al Nat Commun. 2019 10:3660 and Zheng et al Nat Commun. 2017 8: 14049) and may be readily adapted for use herein.

In some embodiments, after the nucleic acid has been capture and optionally barcoded, steps (c)-(e) of the method may be done by: (i) performing a first primer extension reaction using the tethered nucleic acid molecules as a template to produce a first set of primer extension products; (ii) eluting the first set of primer extension products from the tethered nucleic acid molecules; (iii) after step (ii), performing a second primer extension reaction using the tethered nucleic acid molecules as a template to produce a second set of primer extension products; (iv) eluting the first set of primer extension products from the tethered nucleic acid molecules; (v) after step (ii), analyzing the first set of primer extension products using a first method to produce a first data set; (vi) after step (iv), analyzing the second set of primer extension products using a second method to produce a second data set; and (vii) integrating the first and second data sets. Any barcodes/indexes used may be added to the nucleic acid prior to tethering it to the support or the barcodes/indexes may be in the primers such that the complement of the barcode/index will be in the primer extension products. Depending on the source of the nucleic acid (e.g., whether it is genomic DNA or cDNA) and intended use, the primers may be universal (e.g., oligodT), gene-specific, or random, for example. In these embodiments, the cDNA and/or genomic libraries may be further tagged (i.e., in addition to a cell-specific barcode) with unique molecular index (UMI) that identifies individual molecules.

The data sets may be integrated using a discrete sequence element that is present in the sample. For example, the discrete sequence element may be a cell-specific barcode that uniquely identifies a single cell. In these embodiments, the different data sets may be integrated on a cell-by-cell basis. In some embodiments, the discrete sequence element may be a unique molecular index (UMI) that identifies individual nucleic acid molecules in these embodiments, the different data sets may be integrated on a molecule-by-molecule basis (in addition to a cell-by-cell bases, if a cell barcode is used). The discrete sequence element may also be a CRISPR-generated edit, a CRISPR guide RNA sequence, one or more sequence variations (e.g., SNPs) or a splicing isoform defined by a particular combination of exons.

The primer extension products produced in the method may be pooled prior to analysis in step (d). The primer extension products may be analyzed using a variety of different methods, including short read sequencing methods and long read sequencing methods, which produce different types of information. For example, one of the sets of primer extension products may be analyzed by any one or more of the following short read sequencing methods: e.g., Illumina's reversible terminator method, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps. In some embodiments, another of the sets of primer extension products may be analyzed by a long-read sequencing platform such as by nanopore sequencing (e.g. as described in Soni et al Clin Chem 53: 1996-2001 2007, or as described by Oxford Nanopore Technologies). Nanopore sequencing is a single-molecule sequencing technology whereby a single molecule of DNA is sequenced directly as it passes through a nanopore. A nanopore is a small hole, of the order of 1 nanometer in diameter. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology as disclosed in U.S. Pat. Nos. 5,795,782, 6,015,714, 6,627,067, 7,238,485 and 7,258,838 and U.S. patent publications US2006003171 and US20090029477. Alternatively, the primer extension products could be analyzed using Pacific Biosciences' fluorescent base-cleavage method (see, e.g., Chaisson et al Nature 2014 517: 608-611). The different analysis methods of (d) can comprise any one or more of: copy number analysis, analysis of single nucleotide variations (SNVs), analysis of gene expression levels, analysis of isoform structure, analysis of CRISPR edits; analysis of nucleosome position and analysis of epigenetic changes (e.g., methylation), among other things. For example, the primer extension products are analyzed by: copy number analysis, SNV analysis and methylation analysis; gene expression analysis, isoform analysis and methylation analysis; analysis of CRISPR edits, gene expression analysis and isoform analysis; and/or analysis of nucleosome position and methylation analysis.

In certain cases, the nucleic acid sample may be genomic DNA or cDNA (e.g., full length cDNA). If the sample if genomic DNA, it may be produced from genomic DNA using chemical, physical, restriction enzyme or transposase-catalyzed fragmentation methods, see, e.g., Adey et al (Genome Biology 2010, 11:R119). For example, the physical fragmentation methods may be sonication, nebulization, or shearing of genomic DNA. In certain embodiments, prior to performing the method, genomic DNA may be fragmented to an average size in the range of 100 bp to 10 kb, e.g., 200 bp to 5 kb. In certain embodiments, a subject reaction mix may further contain a nucleic acid sample. In particular embodiments, the sample may contain genomic DNA or an amplified version thereof (e.g., genomic DNA amplified by WGA using the Lage method (Lage et al, Genome Res. 2003 13: 294-307), “MDA” (Dean et al Proc. Natl. Acad. Sci. 2002 99: 5261-5266 and Nelson Biotechniques 2002 Supp1:44-47) or by multiple annealing and looping based amplification cycles (“MalBac”; see Zong et al Science. 2012 338: 1622-1626), for example. In exemplary embodiments, the genomic sample may contain genomic DNA from a mammalian cell, such as, a human, mouse, rat, or monkey cell. The sample may be made from cultured cells or cells of a clinical sample, e.g., a tissue biopsy, scrape or lavage or cells of a forensic sample (i.e., cells of a sample collected at a crime scene). In particular embodiments, the genomic sample may be from a formalin fixed paraffin embedded (FFPE) sample.

In particular embodiments, the nucleic acid sample may be obtained from a biological sample such as cells, tissues, bodily fluids, and stool. Bodily fluids of interest include but are not limited to, blood, serum, plasma, saliva, mucous, phlegm, cerebral spinal fluid, pleural fluid, tears, lacteal duct fluid, lymph, sputum, cerebrospinal fluid, synovial fluid, urine, amniotic fluid, and semen. In particular embodiments, a sample may be obtained from a subject, e.g., a human, and it may be processed prior to use in the subject assay. For example, the nucleic acid may be extracted from the sample prior to use, methods for which are known. cDNA may be made from RNA using known method.

The primer extension reaction using the tethered nucleic acid molecules as a template is performed in column, such that the reaction is done while the nucleic acid molecules are still tethered to the support. As would be apparent, this step of the method involves hybridizing a primer to the tethered nucleic acid, adding other necessary reagents, (e.g., polymerase, buffer and nucleotides), and then incubating the product under conditions suitable for primer extension. Primer extension product is produced by this reaction. In some embodiments, the primers used for primer extension may be gene specific in that they hybridize to a subset of the nucleic acid molecules that are tethered to the porous support. In some embodiments, the primer extension step may use a single gene specific primer to copy a single sequence (including any variants thereof) from the sample. In other embodiments, the primer extension step may use multiple gene specific primers to copy several sequences (including any variants thereof) from the sample. In other embodiments, a universal primer, i.e., a primer that is designed to hybridize to all tethered sequences may be employed. Depending on the desired result, the gene specific primers may be pathogen-specific primers (where each primer only primes in the genome of a particular pathogen) or locus-specific (where each primer only primes in the genome of the organism under study, e.g., the human genome). In any of these embodiments, the polymerase may proceed towards the support, or away from the support.

In many embodiments, the primer extension products are eluted from the porous support, while leaving the template tethered to the porous support, to produce an eluted primer extension product. This step may be done by treating the product of the primer extension step with heat (e.g., a temperature of at least 90° C.) or a chaotrophic agent (e.g., sodium iodide, sodium perchlorate, formamide, guanidinium thiocyanate or guanidinium hydrochloride) to denature the primer extension products from the tethered nucleic acid molecules; and applying a force that separates the primer extension products and the porous support. In one embodiment, the force may be a centrifugal force. However, other methods may be used.

After the primer extension product has been eluted from the support, a second primer extension reaction may be done using the same tethered nucleic acid molecules as a template to produce second primer extension products. The support—and the tethered nucleic acid molecules—may be reused several times. The different primer extension products are analyzed using different, orthogonal, methods to provide different types of information about the tethered nucleic acid molecules.

In certain embodiments, the initial DNA being analyzed may be derived from a single source (e.g., a single organism, virus, tissue, cell, subject, etc.), whereas in other embodiments, the nucleic acid sample may be a pool of nucleic acids extracted from a plurality of sources (e.g., a pool of nucleic acids from a plurality of organisms, tissues, cells, subjects, etc.), where by “plurality” is meant two or more. As such, in certain embodiments, a nucleic acid sample can contain nucleic acids from 2 or more sources, 3 or more sources, 5 or more sources, 10 or more sources, 50 or more sources, 100 or more sources, 500 or more sources, 1000 or more sources, 5000 or more sources, up to and including about 10,000 or more sources. Molecular barcodes may allow the sequences from different sources to be distinguished after they are analyzed. In addition, the reaction may be multiplexed such that a plurality of different target loci (e.g., 10 to 1000) are targeted in a single reaction. In these embodiments, the samples may contain a molecular barcode in order to identify the source of a nucleic acid molecule after it is sequenced. In some cases, the barcode is contained within the oligonucleotide 4, which is linked to the nucleic acid molecules at the beginning of the method.

The method described above can be employed to manipulate and analyze DNA from virtually any nucleic acid source, including but not limited to genomic DNA and complementary DNA (cDNA), plasmid DNA, mitochondrial DNA, synthetic DNA, and BAC clones etc. Furthermore, any organism, organic material or nucleic acid-containing substance can be used as a source of nucleic acids to be processed in accordance with the present invention including, but not limited to, plants, animals (e.g., reptiles, mammals, insects, worms, fish, etc.), tissue samples, bacteria, fungi (e.g., yeast), phage, viruses, cadaveric tissue, archaeological/ancient samples, etc. In certain embodiments, the initial DNA used in the method may be derived from a mammal, where in certain embodiments the mammal is a human.

The method described above finds use in a variety of applications, where such applications generally include sample analysis applications in which the presence of a target nucleic acid sequence in a given sample is detected. Because certain embodiments of the method are capable of producing a copy of a sequence in sample, the method finds particular use in targeted resequencing applications in which one or more loci within a genome are selected and then sequenced.

In order to further illustrate the present invention, the following specific examples are given with the understanding that they are being offered to illustrate the present invention and should not be construed in any way as limiting its scope.

### EXAMPLES

The following examples are put forth so as to provide those of ordinary skill in the art with additional disclosure and description of how to make and use the present invention, and are not intended to limit the scope of what the inventors regard as their invention nor are they intended to represent that the experiments below are all or the only experiments performed.

As a demonstration of the APEX method, single cell transcriptome sequencing applications were analyzed. The 10× Genomics platform was used to generate a single cell library, but other single-cell methods (e.g. Drop-Seq) and others should be compatible. As a model system, human lymphocytes were analyzed. These cells are well characterized in single-cell literature and contain vast amounts of human variation with significant implications in human disease. To demonstrate the power of APEX to integrate multiple omic features in single cell transcriptomes, APEX technology was used to directly genotype CRISPR-derived mutations in a polyclonal cell line and integrate such data in the larger context of whole transcriptome data.

### METHODS

Single cell processing: The MMNK1 cell line was obtained from JCRB (Japan). Cells were cultured in their recommended media conditions at 37° C. Cultured cells were processed into suspensions with standard procedures. Briefly, the cell line was trypsinized, followed by inactivation by FBS. The cells were washed by centrifugation at 400 g in 1×PBS with 0.04% BSA. To remove cellular debris and cellular aggregates, the cells were filtered through a Flowmi cell strainer (Wayne, N.J.) before proceeding to single-cell RNA processing. Blood collected in EDTA or sodium heparin was overlaid on 15 ml Ficoll-Paque Plus (GE healthcare, Chicago, Ill.) contained in a Sepmate 50 ml tube (Stemcell Technologies, Vancouver, Canada), and centrifuged for 10 minutes at 1200 g. Interphase containing the PBMCs was decanted into a fresh tube followed by 2 washes with 1×PBS with centrifugation at 400 g for 3 minutes.

Single cell library generation: Single cell libraries were generated using the 10× Genomics Chromium system using the Library and Gel Bead Kit (either 5′ or 3′ kits). Briefly, single cell suspensions were loaded alongside enzymatic reagents into a microfluidic chip according to manufacturer's protocols, with 14 cycles of PCR during the full-length cDNA amplification process. A portion of this material was carried through to Illumina library preparation according to manufacturer's protocols, and the rest was saved for APEX conjugation. Briefly, Illumina library preparation consists of enzymatic fragmentation, followed by end repair and a-tailing and ligation of the fragments with a second Illumina sequencing adapter. The libraries were quantified with Qubit (Thermo Fisher Scientific) and sequenced on an Illumina sequencer. Standard cellranger processing workflows, offered as a part of the official 10× Genomics computational pipeline, was used to demultiplex the sequencing data and quantify single cell gene expression. Clustering and tSNE visualization was performed with Seurat.

APEX conjugation of full length cDNA: Full length single-cell cDNA was first denatured by heat shock at 95 C for 5 minutes, followed by immediately ramping down to 4 C on ice or in a PCR machine. The resultant DNA was then tailed with TCO groups by incubation in 1× Terminal Transferase Buffer (New England Biolabs), 1 ul terminal transferase (New England Biolabs), and 5 ul 1 mM TCO-PEG4-dUTP (Jena Biosciences) at 37 C for one hour without heat inactivation. The resultant mixture was then purified with Ampure XP beads (Beckman Coulter) using a 1.8× ratio of beads to sample volume according to the manufacturer's protocols. The DNA was then eluted in elution buffer (10mM Tris-HCl pH 8.0, 0.05% Tween-20). During the cleanup process, 50 ul of tetrazine-functionalized magnetic agarose beads (Cube Biotech; custom ordered) was transferred to another microtube and washed twice with elution buffer. After washing, the eluted DNA was then transferred to the magnetic agarose beads. The beads were resuspended, and then placed on a rotary shaker at room temperature overnight. The next day, conjugation efficiency can be measured using Qubit fluorimetry by transferring 20 ul of the supernatant (after magnetization) to a single Qubit dsDNA HS reaction. The beads are then washed twice using elution buffer, and can be stored at −20 C in storage buffer (50% glycerol, 10 mM Tris-HCl pH 8.0, 0.5% Tween-20).

Whole transcriptome amplification with APEX conjugated DNA: APEX conjugated single cell cDNA is removed from −20 C and is washed twice with elution buffer before proceeding with next steps. Whole transcriptome amplification is performed using 500 nM PCR primers corresponding to a Partial Read 1 sequence (CTACACGACGCTCTTCCGATCT; SEQ ID NO: 2), and either the 5′ flanking primer (AAGCAGTGGTATCAACGCAGAG; SEQ ID NO: 3) or the 3′ flanking primer (AAGCAGTGGTATCAACGCAGAGTACAT; SEQ ID NO: 4) as described in the official single cell protocol supplied by 10× Genomics, and 1× Kapa HotStart ReadyMix (Roche) resuspended into the bead mixture. Cycling conditions are: 95 C for 45 seconds, followed by 14 cycles of: 95 C for 15 seconds, 60 C for 30 seconds, and 72 C for 30 seconds, and a final extension at 72 C for 30 seconds before infinite hold at 4 C. After amplification, the beads are magnetized and supernatant transferred to another tube. The beads are washed twice by incubation at 65 C for 1 minute with wash buffer (98% DMSO, 10 mM Tris-HCl pH 8.0, 1% Tween-20), followed by magnetization, removal of wash buffer, resuspension in storage buffer, and storage at −20 C. The amplification products are purified with Ampure XP beads using 1.8× beads to sample ratio using standard protocols.

Nanopore sequencing: Amplicon products were quantified by Qubit and visualized on an E-gel (Thermo Fisher Scientific) to calculate an approximate molecular concentration of the sample. Approximately 300 fmol were carried forward for nanopore sequencing library preparation. Our protocol is based on the official SQK-LSK109 library preparation kit by Oxford Nanopore Technologies (United Kingdom). Briefly, end repair and a-tailing was performed using the Kapa HyperPrep library preparation kit (Roche). DNA purification was done using Ampure XP beads with 1.8× beads to sample ratio, and eluted the DNA into 60 ul of 10mM Tris-HCl pH 8.0 buffer. Afterward, adapter ligation was done using 25 ul LNB buffer (Oxford Nanopore), 5 ul AMX adapter (Oxford Nanopore), 10 ul Kapa HyperPrep ligase (Roche), and incubated for 15 minutes at room temperature. The sample was then purified with 1.5× ratio of Ampure XP beads to sample, but washed twice with SFB buffer (Oxford Nanopore) where the beads were briefly resuspended by flicking the tube. The beads were eluted for one hour with 12.5 ul EB buffer (Oxford Nanopore). The library was then loaded into the Oxford Nanopore MinION flowcell using standard procedures and sequenced for 48 hours.

Bioinformatic processing and data integration: Nanopore sequence data was basecalled using Guppy (v3.2.1), the official basecalling software supported by Oxford Nanopore. The resultant FASTQs were then split into reads containing the cellular barcode-UMI region and the cDNA region by using cutadapt. Cutadapt can split nanopore reads using an internal adapter sequence. Subsequent cDNA sequence reads were aligned with minimap2.

Datasets from multiple APEX assays can be integrated by discrete elements such as cellular barcodes. To integrate short read and long read transcriptome datasets together, cellular barcodes were matched between datasets. A ground truth set of cellular barcodes for an experiment was determined by cellranger on an Illumina sequencing run of a single cell library. Cellular barcodes in the nanopore sequence data were determined by a custom implementation of a k-mer matching approach. Briefly, all possible k-mers of a particular length (e.g., 10) were counted for all observed cellular barcodes in the Illumina dataset. This results in a k×n matrix, where k is number of observed k-mers, n is the number of cells, and each entry in the matrix is the count observed. For each nanopore read, the same counting procedure can be performed but only considering k-mers that were observed in the Illumina dataset. Multiplying the two matrices together results in a 1×n vector of scores corresponding to the similarity of each read to a particular cellular barcode. The maximum value is then extracted for barcode assignment.

Cells of desired genotypes can also be assigned a cellular barcode and thus linked to an Illumina dataset. Single cell transcriptome nanopore reads of MMNK1 cells with guide RNA sequences targeting ARID1A were extracted by retrieving reads with soft-clipping at the site of the anticipated base edit. The associated cellular barcode was then assigned and then linked to the tSNE map or assigned cluster from the nanopore dataset.

Target enrichment. Enrichment of specific transcripts was performed by designing primers complementary to the last exon of a specific gene. To design primers, design principles as previously described that measure for a given 20-mer sequence the number of matches across the human genome were used. All the possible tiling 20-mer sequences were generated from the last exon of a gene of interest, and then calculate the number of perfect matches, number of matches with a single mismatch, and the number of matches with exactly two mismatches across the human genome. To select a primer sequence, a 20-mer sequence that has only one perfect match across the human genome wsa picked, zero matches with a single mismatched base, and the least matches with a double mismatch for all candidates in the exon. Oligonucleotides were then ordered from IDT.

To perform enrichment, these primers were then combined as a pool with the Partial Read 1 sequence (CTACACGACGCTCTTCCGATCT; SEQ ID NO: 5). APEX conjugated beads were removed from storage, and washed twice with elution buffer. An amplification reaction was then performed with 500 nM of the primer pool and 1× KAPA HotStart ReadyMix with the following conditions: 98 C for 45 C, 14 cycles of 98 C for 15 C, 55 C for 30 C, and 72 C for 30 C, followed by a final hold at 72 C for 1 minute and infinite hold at 4 C. The beads were then magnetized and the PCR products transferred to another PCR tube. The beads are washed twice by incubation at 65C for 1 minute with wash buffer (98% DMSO, 10 mM Tris-HCl pH 8.0, 1% Tween-20), followed by magnetization, removal of wash buffer, resuspension in storage buffer, and storage at −20 C. The amplification products are purified with Ampure XP beads using 1.8× beads to sample ratio using standard protocols. Afterward, the amplified products can then be processed with nanopore library preparation protocols as described above.

### RESULTS

### Full Length Single-Cell cDNA Conjugation Via ‘Click Chemistry’

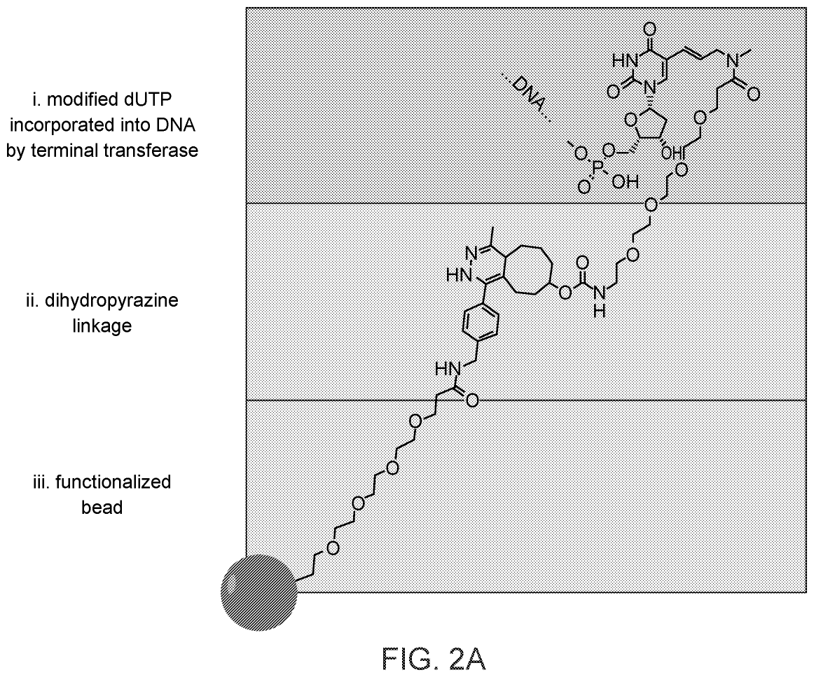

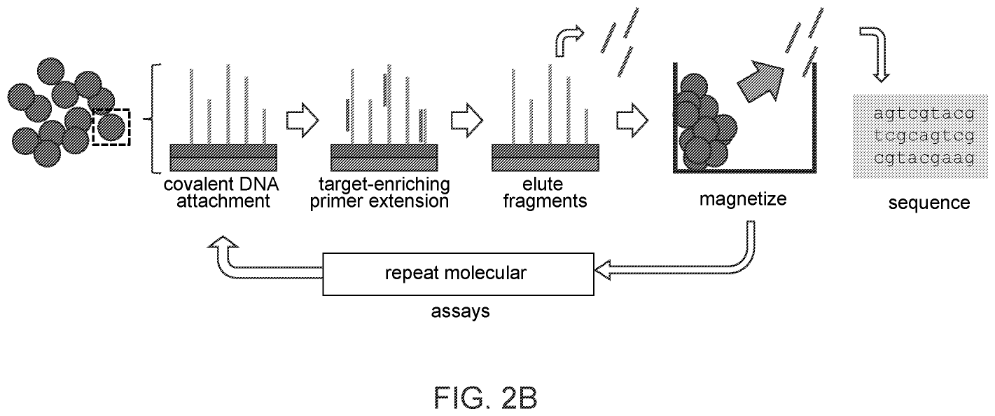

‘Click chemistry’ was used to conjugate DNA onto magnetic agarose beads (FIG. 2A). Full-length cDNA is tagged with single cell barcodes using the 10× Genomics platform. APEX is compatible with any other single cell methodology, since nearly all scRNA-Seq methods convert mRNA into cDNA molecules. Subsequent to conjugation, repeated molecular assays using polymerases can be performed (FIG. 2B).

‘Click chemistry’ is a type of ‘green’ chemistry that enables rapid and quantitative conjugation between molecular species in mild biocompatible buffers. The specific ‘click chemistry’ variant used in this study is the inverse demand Diels-Alder cycloaddition (iEDDA) that occurs between tetrazines (Tz) and trans-cyclooctenes (TCO). This approach has rapid reaction rates even at biological sample concentrations (e.g. 10 nanograms of 1 kb cDNA is ˜15 fmol of material). iEDDA-based click chemistry uses biocompatible buffers and does not compromise cDNA integrity.

A streamlined protocol to conjugate DNA to functionalized magnetic beads was developed (FIG. 2C). Terminal transferase non-specifically adds TCO-modified nucleotides to single-cell cDNA libraries, and incubation with Tz-modified magnetic beads leads to chemical conjugation. A >90% conjugation efficiency with small molecules (TCO-Cy5), TCO-modified oligonucleotides, and TCO-tailed single-cell cDNA. The retention of conjugated molecules was tracked using molecular barcodes. Over the course of seven experiments spanning three months, molecular barcode diversity was maintained, enabling repeated and iterative analyses. Thus, iterative experiments on conjugated single cell cDNA, such as measuring both short and long read transcriptome profiles, are enabled with APEX technology.

### Iterative Short- and Long-Read Sequencing with APEX.

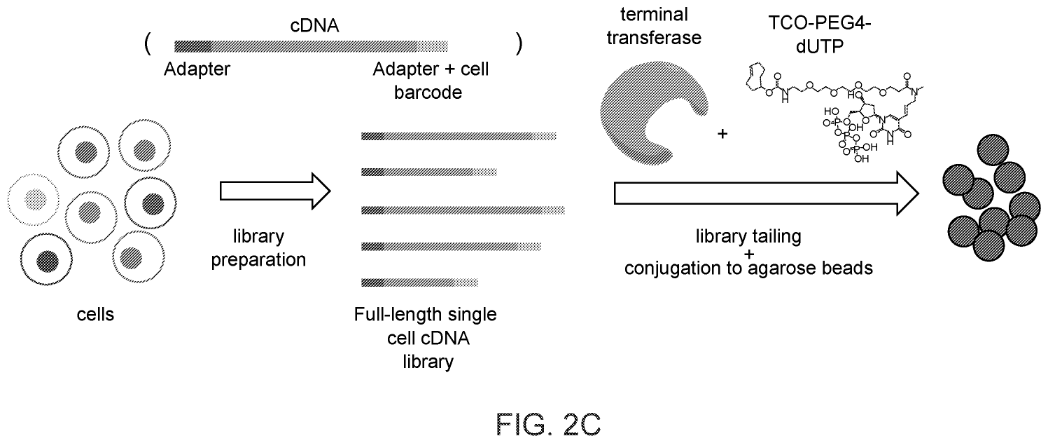

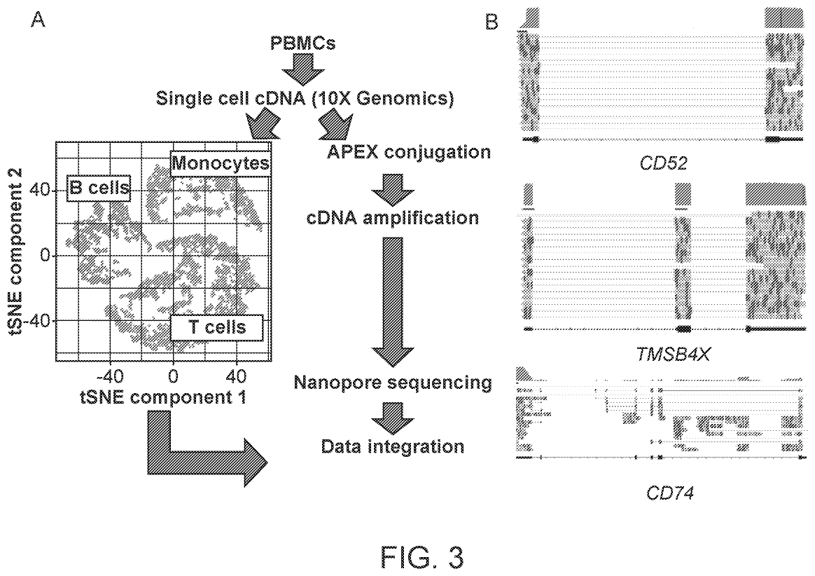

One application of APEX is the simultaneous analysis of single-cell transcript levels (Illumina short reads) and differential splicing (PacBio or ONT long reads). Single cell cDNA from blood-derived leukocytes was studied using multiple sequencing technologies (FIG. 3A). Conjugated full-length cDNA from single cells was prepared with a 10× Genomics Chromium instrument. The input material consists of flanking adapters that enable amplification of full-length cDNA as well as the barcode sequence associated with each cell.

As a control, results between standard single cell RNA-Seq were compared with short reads and APEX conjugation for long reads. Full-length cDNA was amplified from the DNA-conjugated magnetic beads and performed ONT sequencing (FIG. 3B). Over 10,000 genes were detected, as well as differentially expressed genes and isoforms among cell types.

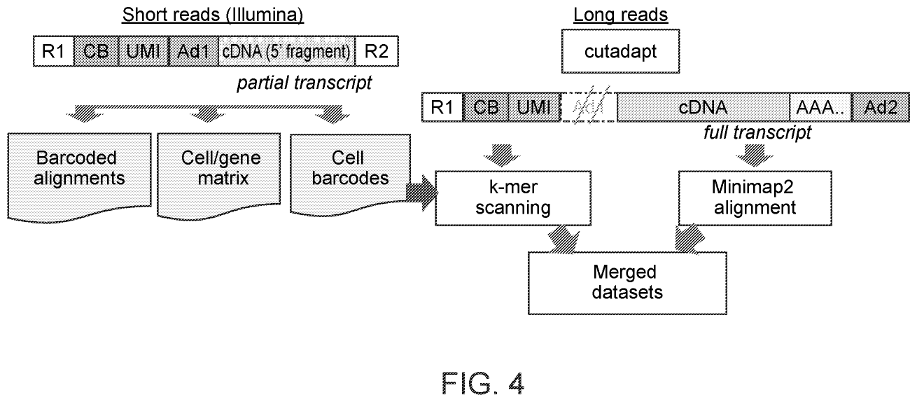

APEX enables the measurement of multiple omic features from the same molecule. With APEX, both long and short read sequencing can be performed on the cDNA libraries derived from the same transcript molecules. To integrate short and long read datasets, a bioinformatic workflow consisting of open source software and custom-built python scripts was developed (FIG. 4A). As sequencing errors in ONT long reads prevent efficient edit distance-based assignment of cellular barcodes to a ground truth based on short reads, a novel approach was developmed to match k-mer sequences between datasets similar to methods used in natural language processing. Briefly, a k-mer profile was generated for each detected cellular barcode in the short-read dataset. For each cellular barcode sequence region in the noisy long-read dataset, a k-mer profile was also generated. To match a noisy long-read cellular barcode to one in the short-read data, a simple cosine distance was determined through matrix multiplication.

This simple k-mer matching method was very robust in reducing the impact of non-informative k-mers in the Read 1 and UMI sections and enabled us to resolve cellular barcodes for practically all reads. The majority of reads had a cosine similarity of ˜0.8 with an observed Illumina-derived barcode sequence; this is several-fold higher than other studies using nanopore single cell RNA sequencing. Additional tools were developed to extract full-length alignments from transcripts of interest, to collate their associated cellular barcodes, and to link this information with Illumina short read data. Integrating full-transcript long read, short-read higher depth alignments and per-cell counting provided a comprehensive dataset linking single cell transcript isoform usage and short-read transcriptomes.

### Data Integration Using Discrete Elements

The vast amount of omic features in single cell datasets can be linked by discrete features such as cell barcodes, or features such as CRISPR edits, molecular barcodes and SNPs. In CRISPR-based perturbation assays such as Perturb-Seq and CROP-Seq, the guide RNA sequence is used to identify single cells but does not provide the CRISPR edit genotype. In contrast, long-read sequencing coupled with short-read data was used to directly detect CRISPR-induced mutations. With this technology the genome edit introduced in an engineered transcript isoform was directly read as opposed to simply sequencing a guide RNA barcode.

This feature enables us to call CRISPR-related single nucleotide variants (SNVs), frameshift, and non-frameshift insertion-deletions (indels). As CRISPR edits are not introduced with 100% efficiency, direct determination of the edited genotype enables the separation of successfully edited cells from unmodified genomic sites at single cell resolution and across millions of sites, once scaled up.

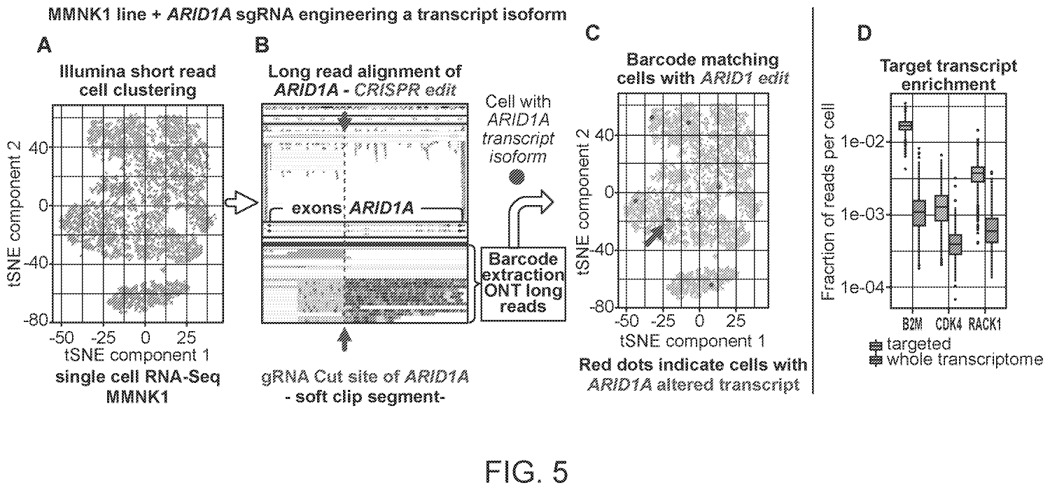

As another demonstration of APEX, cross-platform data integration of a CRISPR-edited cell line (FIG. 5) was performed. The MMNK1 cholangiocyte cell line was transfected with plasmids encoding Cas9 and guide RNAs targeting the gene ARID1A. The polyclonal cell line was made into single cell cDNA and APEX conjugated onto magnetic beads. Chort-read sequencing was conducted on a portion of this library with an Illumina system (FIG. 5A). The remaining library was conjugated to magnetic beads, amplified using flanking primers, and performed nanopore sequencing. Improvements in library preparation protocols and base calling with long reads above enabled highly accurate assignment of nanopore reads to cellular barcodes. Focusing on the designed edit site, cells with an edit (that did not trigger nonsense-mediated decay) were identified using barcode matching (FIG. 5B). The data was integrated with quantitative expression data as well as their place in tSNE space (FIG. 5C), enabling direct correlation of edited genotypes to transcriptional profiles as opposed to simple guide RNA barcode counting.

### Target Enrichment of Selected Genes of Interest

By virtue of covalent attachment, APEX enables facile targeting of single molecules or omic features for iterative analysis. APEX enables a streamlined workflow for targeted sequencing of specific single-cell transcripts. Single primer extension and amplification protocols were used, which are compatible with DNA molecules conjugated onto magnetic beads with multiplexing capacity for tens of thousands of targets. A bioinformatic pipeline was developed for primer design that accounts for melting temperature, degree of uniqueness in the human genome, the proximity of known SNPs, etc. The pipeline determines the uniqueness of a candidate primer sequence across the human genome, by measuring whether a given sequence has: any exact matches; matches with single mismatches; and matches with double mismatches. Candidate primers were selected that minimize matches elsewhere in the human genome.

To experimentally demonstrate target transcript enrichment on single-cell cDNA, targeting primers were designed for several genes of interest and apply them to the conjugated MMNK1 cell line. In a multiplexed pool, transcript-specific amplification was performed with primers that hybridize to target exons and a common flanking primer corresponding to an adapter sequence. The eluted products were then sequenced (FIG. 5D), which yielded significant enrichment rates for transcripts of interest.

### REFERENCES

1 Goetz, J. J. & Trimarchi, J. M. Transcriptome sequencing of single cells with Smart-Seq. Nature biotechnology 30, 763-765, doi:10.1038/nbt.2325 (2012).
2 Macosko, E. Z. et al. Highly Parallel Genome-wide Expression Profiling of Individual Cells Using Nanoliter Droplets. Cell 161, 1202-1214, doi:10.1016/j.cell.2015.05.002 (2015).
3 Zheng, G. X. et al. Massively parallel digital transcriptional profiling of single cells. Nat Commun 8, 14049, doi:10.1038/ncomms14049 (2017). PMCID: PMC5241818 L. M., D. A. M., S. Y. N., M. S. L., P. W. W., C. M. H., R. B., A. W., K. D. N., T. S. M. and B. J. H. are employees of 10× Genomics.
4 Zhang, L. et al. Single-cell whole-genome sequencing reveals the functional landscape of somatic mutations in B lymphocytes across the human lifespan. Proceedings of the National Academy of Sciences of the United States of America 116, 9014-9019, doi:10.1073/pnas.1902510116 (2019). PMCID: PMC6500118
5 Andor, N. et al. Single-cell RNA-Seq of follicular lymphoma reveals malignant B-cell types and coexpression of T-cell immune checkpoints. Blood 133, 1119, doi:10.1182/blood-2018-08-862292 (2019). PMCID: PMC6405336
6 Buenrostro, J. D. et al. Integrated Single-Cell Analysis Maps the Continuous Regulatory

Landscape of Human Hematopoietic Differentiation. Cell 173, 1535-1548.e1516, doi:10.1016/j.cell.2018.03.074 (2018). PMCID: PMC5989727
7 Velten, L. et al. Human haematopoietic stem cell lineage commitment is a continuous process. Nature cell biology 19, 271-281, doi:10.1038/ncb3493 (2017). PMCID: PMC5496982
8 Chen, J. et al. Single-cell transcriptome analysis identifies distinct cell types and niche signaling in a primary gastric organoid model. Scientific Reports 9, 4536, doi:10.1038/s41598-019-40809-x (2019). PMCID: PMC6418230
9 Sathe, A. et al. Single cell genomic characterization reveals the cellular reprogramming of the gastric tumor microenvironment. bioRxiv, 783027, doi:10.1101/783027 (2019).
10 Stenson, P. D. et al. The Human Gene Mutation Database: building a comprehensive mutation repository for clinical and molecular genetics, diagnostic testing and personalized genomic medicine. Hum Genet 133, 1-9, doi:10.1007/s00439-013-1358-4 (2014). PMCID: PMC3898141
11 Gupta, I. et al. Single-cell isoform RNA sequencing characterizes isoforms in thousands of cerebellar cells. Nature biotechnology 36, 1197-1202, doi:10.1038/nbt.4259 (2018).
12 Singh, M. et al. High-throughput targeted long-read single cell sequencing reveals the clonal and transcriptional landscape of lymphocytes. Nature Communications 10, 3120, doi:10.1038/s41467-019-11049-4 (2019). PMCID: PMC6635368
13 Stuart, T. et al. Comprehensive Integration of Single-Cell Data. Cell 177, 1888-1902.e1821, (2019). PMCID: PMC6687398
14 Fan, J. et al. Linking transcriptional and genetic tumor heterogeneity through allele analysis of single-cell RNA-seq data. Genome research 28, 1217-1227, doi:10.1101/gr.228080.117 (2018). PMCID: PMC6071640
15 Lau, B. T. & Ji, H. P. Covalent “Click Chemistry”-Based Attachment of DNA onto Solid Phase Enables Iterative Molecular Analysis. Analytical chemistry 91, 1706-1710, doi:10.1021/acs.analchem.8b05139 (2019).
16 Schmiedel, B. J. et al. Impact of Genetic Polymorphisms on Human Immune Cell Gene Expression. Cell 175, 1701-1715.e1716, doi:10.1016/j.cell.2018.10.022 (2018). PMCID: PMC6289654
17 Karver, M. R., Weissleder, R. & Hilderbrand, S. A. Synthesis and Evaluation of a Series of 1,2,4,5-Tetrazines for Bioorthogonal Conjugation. Bioconjugate Chemistry 22, 2263-2270, doi:10.1021/bc200295y (2011). PMCID: PMC3254257
18 Shin, G. et al. CRISPR-Cas9-targeted fragmentation and selective sequencing enable massively parallel microsatellite analysis. Nature Communications 2017 8, 14291, doi:10.1038/ncomms14291

1. A method for sample analysis, comprising:
(a) incubating a nucleic acid sample with a terminal transferase and a cyclooctene-functionalized nucleotide to produce cyclooctene-functionalized nucleic acid molecules;
(b) tethering the cyclooctene-functionalized nucleic acid molecules to a tetrazine-functionalized support via an Alder cycloaddition reaction;
(c) performing at least two separate primer extension reactions using the tethered nucleic acid molecules as a template to produce multiple distinct sets of primer extension products;
(d) separately analyzing the sets of primer extension products using different methods to produce multiple data sets; and
(e) integrating the data sets.

2. The method of claim 1, wherein:
the nucleic acid sample of step (a) is made by:
i. compartmentalizing a population of cells into single cell compartments;
ii. making cDNA and/or genomic DNA libraries from the cells in the compartments, wherein the cDNA and/or genomic libraries are tagged with different cell-specific barcodes; and
iii. pooling the cDNA and/or genomic libraries made from the cells; and
the integrating of step (e) is done on a cell-by-cell basis.

3. The method of claim 2, wherein the cDNA and/or genomic libraries are further tagged with unique molecular index (UMI) that identifies individual molecules.

4. The method of claim 1, wherein steps (c)-(e) are done by:
(i) performing a first primer extension reaction using the tethered nucleic acid molecules as a template to produce a first set of primer extension products;
(ii) eluting the first set of primer extension products from the tethered nucleic acid molecules;
(iii) after step (ii), performing a second primer extension reaction using the tethered nucleic acid molecules as a template to produce a second set of primer extension products;
(iv) eluting the first set of primer extension products from the tethered nucleic acid molecules;
(v) after step (ii), analyzing the first set of primer extension products using a first method to produce a first data set;
(vi) after step (iv), analyzing the second set of primer extension products using a second method to produce a second data set; and
(vii) integrating the first and second data sets.

5. The method of claim 1, wherein the data sets are integrated using a discrete sequence element that is present the sample.

6. The method of claim 5, wherein the discrete element is one or more of the discrete elements selected from the group consisting of:
a cell-specific barcode that uniquely identifies a single cell,
a unique molecular index (UMI) that identifies individual nucleic acid molecules;
a CRISPR-generated edit;
a CRISPR guide RNA sequence;
one or more sequence variations; and
a splicing isoform defined by a particular combination of exons.

7. The method of claim 1, wherein the method is done on genetic material obtained from a single cell.

8. The method of claim 1, wherein the method is done on genetic material obtained from a population of cells.

9. The method of claim 1, wherein the analysis of (d) comprises short read sequence analysis and long read sequence analysis.

10. The method of claim 1, wherein the different methods of (d) comprise: copy number analysis, analysis of single nucleotide variations (SNVs), analysis of gene expression levels, analysis of isoform structure, analysis of CRISPR edits; analysis of nucleosome position and analysis of epigenetic changes (e.g., methylation).

11. The method of claim 1, wherein the distinct sets of primer extension products are analyzed by:
copy number analysis, SNV analysis and methylation analysis;
gene expression analysis, isoform analysis and methylation analysis;
analysis of CRISPR edits, gene expression analysis and isoform analysis; and/or
analysis of nucleosome position and methylation analysis.

12. The method of claim 1, wherein the cyclooctene-functionalized nucleotide is 5-trans-Cyclooctene-PEG₄-dUTP.

13. The method of claim 1, wherein at least one of the primer extension reaction uses gene specific primers that hybridize to a subset of the nucleic acid molecules that are tethered to the support.

14. The method of claim 1, wherein the primer extension reaction uses universal primers that hybridize to an oligonucleotide that is appended to the nucleic acid molecules.

15. The method of claim 1, wherein the sample comprises genomic DNA or cDNA.

16. The method of claim 1, wherein the sample is obtained from a tissue biopsy.

17. The method of claim 1, wherein the sample is obtained from a patient having an infectious disease or condition.

18. The method of claim 1, wherein the sample is obtained from blood cells.

19. The method of claim 1, wherein the at least two separate primer extension reactions comprise one or more PCR reactions.


SEQUENCE LISTING (5 sequences)

SEQ ID NO: 1 - DNA, 27 bp, Artificial sequence
  synthetic oligonucleotide
agtcgtacgtcgcagtcgcgtacgaag

SEQ ID NO: 2 - DNA, 22 bp, Artificial sequence
  synthetic oligonucleotide
ctacacgacgctcttccgatct

SEQ ID NO: 3 - DNA, 22 bp, Artificial sequence
  synthetic oligonucleotide
aagcagtggtatcaacgcagag

SEQ ID NO: 4 - DNA, 27 bp, Artificial sequence
  synthetic oligonucleotide
aagcagtggtatcaacgcagagtacat

SEQ ID NO: 5 - DNA, 22 bp, Artificial sequence
  synthetic oligonucleotide
ctacacgacgctcttccgatct